(19) United States
(12) Patent Application Publication
(10) Pub. No.: US 2024/0189475 A1
(43) Pub. Date: Jun. 13, 2024

(54) NANOFIBROUS SCAFFOLD COMPRISING USNIC ACID FOR SKIN TISSUE REGENERATION

(71) Applicant: PUKYONG NATIONAL UNIVERSITY INDUSTRY-UNIVERSITY COOPERATION FOUNDATION, Busan (KR)

(72) Inventors: Won-Kyo Jung, Busan (KR); Milan Pathum Chandika, Busan (KR); Tae-Hee Kim, Busan (KR); Se-Chang Kim, Gyeongju-si (KR)

(73) Assignee: PUKYONG NATIONAL UNIVERSITY INDUSTRY-UNIVERSITY COOPERATION FOUNDATION, Busan (KR)

(21) Appl. No.: 18/316,623

(22) Filed: May 12, 2023

(30) Foreign Application Priority Data
Dec. 13, 2022 (KR) 10-2022-0173566

(51) Int. Cl. A61L 15/26; A61L 15/40; A61L 15/42; A61L 27/18; A61L 27/36; A61L 27/50

(52) U.S. Cl. CPC A61L 15/26; A61L 15/40; A61L 15/42; A61L 27/18; A61L 27/3633; A61L 27/50; B82Y 5/00

(57) ABSTRACT

An example embodiment of the present disclosure relates to a nanofiber scaffold including polycaprolactone (poly(ε-caprolactone; PCL), decellularized extracellular matrix (dECM) and usnic acid (UA), and a preparation method thereof. The nanofiber scaffold of an example embodiment of the present disclosure has a uniform nanofiber structure, good fluid absorption and retention, moderate release rate, high mechanical stability, thermal stability, and semi-crystalline structure, and exhibits excellent antibacterial efficacy against fungi/bacteria such as Candida albicans, and good compatibility with human-derived fibroblasts.

### CROSS-REFERENCE TO RELATED APPLICATIONS

This application is based on and claims priority from Korean Patent Application No. KR10-2022-0173566, filed on Dec. 13, 2022, with the Korean Intellectual Property Office, the disclosure of which is incorporated herein in its entirety by reference.

### TECHNICAL FIELD

The present disclosure relates to a nanofiber scaffold including polycaprolactone (poly(ε-caprolactone; PCL), decellularized extracellular matrix (dECM) and usnic acid (UA), and a preparation method thereof.

### BACKGROUND

A microbial community, biofilm formation and subsequent infection may produce various substances, including toxins, which impede wound healing and complicate a regeneration process. A scaffold for skin regeneration fabricated to avoid wound infection and eradicate infected pathogens needs to have excellent antibacterial activity.

Usnic acid (UA) is a secondary metabolite of sea moss and is a molecule with strong antibacterial activity against various pathogens. The UA also has significant potential in the fields of pharmacology and tissue engineering applications due to its unique biological and physiological activities such as antioxidant, anti-inflammatory, antiviral, fibroblast migration, growth factor release enhancement and wound healing properties. However, due to its low solubility in water, its usability is somewhat limited. To improve a delivery range, the UA is mounted to various delivery systems for utilization such as liposomes, films, and nanoparticles.

Decellularization is a technique to remove cells while maintaining important physiologically active molecules such as glycosaminoglycans, collagens, proteoglycans and growth factors and core morphological, structural, mechanical and biochemical properties. Decellularized extracellular matrix (dECM) is widely used in tissue engineering due to its biocompatibility, unique properties, and numerous biological properties. However, due to the loss of structural and mechanical properties and the increase in solubility, the dECM is not applied alone to the fabrication of nanofiber scaffolds for skin regeneration.

Nanofibers fabricated utilizing electrospinning technology have similar structural properties to natural ECM in terms of their interconnected porous structure, and thus have attracted considerable attention in the field of skin tissue engineering applications. Electrospinning may be used with a variety of synthetic polymers, including polycaprolactone (PCL), due to their unique properties such as mechanical properties, biodegradability and biocompatibility. The PCL is a widely used semi-crystalline aliphatic polyester approved for biomedical use by the U.S. Food and Drug Administration (FDA). However, due to its hydrophobicity and low response to cells, the PCL is mixed and used with various natural polymers such as collagen, gelatin, alginate, and chitosan to control biological and mechanical properties and provide structural functions suitable for tissue regeneration.

### SUMMARY

The inventors of the present disclosure prepared polycaprolactone-based nanofibers utilizing usnic acid and pig skin-derived decellularized extracellular matrix and identified their structural properties, antibacterial activity, and skin regeneration ability to complete the present disclosure.

Accordingly, an example embodiment of the present disclosure provides a nanofiber scaffold including polycaprolactone (poly(ε-caprolactone; PCL), decellularized extracellular matrix (dECM) and usnic acid (UA), and a preparation method thereof.

According to the example embodiment of the present disclosure, there is provided the nanofiber scaffold including the PCL, the dECM, and the UA.

In addition, according to another example embodiment of the present disclosure, there is provided a method of preparing a nanofiber scaffold, in which the method includes: mixing polycaprolactone, decellularized extracellular matrix, and usnic acid (phase 1); preparing a nanofiber mat by electrospinning the mixture (phase 2); cross-linking the nanofiber mat (phase 3); and freeze-drying the cross-linked nanofiber mat (phase 4).

In addition, according to yet another example embodiment of the present disclosure, there is provided a wound dressing including the nanofiber scaffold according to the example embodiment of the present disclosure.

The PCL/dECM/UA nanofibers for skin regeneration of an example embodiment of the present disclosure may be fabricated utilizing electrospinning technology, which enables commercial production. In addition, the nanofiber of an example embodiment of the present disclosure has a uniform nanofiber structure, good fluid absorption and retention, moderate release rate, high mechanical stability, thermal stability, and semi-crystalline structure, and exhibits excellent antibacterial efficacy against Candida albicans and four other fungi/bacteria, and good compatibility with human-derived fibroblasts. It was identified through an in vivo full-thickness incisional wound healing mouse model that the fabricated hydrogels significantly improved wound closure by day 21, and histomorphometric evaluation of wound site tissues revealed rapid re-epithelialization, differentiated dermis and epidermis with minimal scar tissue remaining. Accordingly, the fabricated nanofibers exhibited excellent antibacterial activity and skin regeneration efficacy, making them applicable for wound healing applications.

### BRIEF DESCRIPTION OF THE DRAWINGS

FIG. 1 shows the results of morphological analysis of polycaprolactone (PCL), polycaprolactone/usnic acid (PCLU), and polycaprolactone/usnic acid/decellularized extracellular matrix (PEU) nanofibers by mixing ratio: (A) 5 k× and (B) 15 k×-magnified SEM images of the nanofibers and (C) corresponding nanofiber diameter distribution (frequency distribution), (D) 5 k× and (E) 15 k×-magnified SEM images of the nanofibers after cross-linking and (F) corresponding nanofiber diameter distribution, (G) pore size distribution, and (H) porosity of the cross-linked scaffolds.

FIG. 2 is a result of analyzing the physicochemical properties of polycaprolactone (PCL), polycaprolactone/usnic acid (PCLU), and polycaprolactone/usnic acid/decellularized extracellular matrix (PEU) nanofibers by mixing ratio: (A) FTIR spectra, (B) X-ray diffraction profiles, (C) thermogravimetric analysis, (D) differential scanning calorimetry of pure PCL, dECM, and UA and PCLU and PEU nanofiber scaffolds. (E) Mechanical characterization results of nanofiber scaffolds: (a) typical stress-strain, (b) tensile strength, (c) strain at maximum load, and (d) strain at break.

FIG. 3 is a result of analyzing the hydrophilicity, swelling properties, degradation rate and drug release amount of polycaprolactone (PCL), polycaprolactone/usnic acid (PCLU), and polycaprolactone/usnic acid/decellularized extracellular matrix (PEU) nanofibers by mixing ratio: (A) Photograph and change, (B) change over 12 seconds, (C) swelling properties from 0 to 48 hours, (D) swelling properties from 0 to 2 hours, (E) degradation rate, and (F) UA release profile of nanofiber PCL, PCLU, and four different PEU nanofiber scaffolds.

FIG. 4 is a graph showing (A) the results of a disk diffusion antibacterial test to identify the antibacterial efficacy and (B) the inhibition zone against bacterial and fungal pathogens of polycaprolactone (PCL), polycaprolactone/usnic acid (PCLU), and polycaprolactone/usnic acid/decellularized extracellular matrix (PEU) nanofibers by mixing ratio.

FIG. 5 is a graph showing (A) photographs of agar plates showing the appearance and disappearance of microbial cells incubated with different types of nanofiber scaffolds and (B) the reduction in visible microbial cells.

FIG. 6 is a result of analyzing the biofilm formation inhibitory efficacy of polycaprolactone (PCL), polycaprolactone/usnic acid (PCLU), and polycaprolactone/usnic acid/decellularized extracellular matrix (PEU) nanofibers by mixing ratio: (A) Photographs of agar plates showing the appearance and disappearance of microbial cells exposed to the nanofiber scaffolds during biofilm formation of P. aeruginosa and K. pneumoniae. (B) Graphs showing the biofilm inhibition rates of P. aeruginosa and K. pneumoniae. (C) SEM photomicrographs showing the biofilm structures of P. aeruginosa and K. pneumoniae formed on the nanofiber scaffolds.

FIG. 7 shows the results of MTT assay identifying the cytotoxicity and cell proliferation of (A) HDF and (B) HaCaT cells on day 1 and day 5 after seeding on the nanofiber scaffold. (C) Photomicrographs of live/dead cells stained with FDA and PI on day 1 and day 5 after being seeded on the nanofiber scaffold. A tissue culture plate (TCP) is a control.

FIG. 8 is a result of examining the skin regeneration efficacy in a full-thickness incisional wound-induced mouse model of polycaprolactone (PCL), polycaprolactone/usnic acid (PCLU), and polycaprolactone/usnic acid/decellularized extracellular matrix (PEU) nanofibers by mixing ratio: (A) Photographs of full-thickness incisional wound healing of ICR mice treated with nanofiber scaffolds on days 0, 7, 14, and 21 after surgery, and (B) Image J (image analysis software) measurement results showing average wound closure after surgery. (C) Photomicrographs of the H&E-stained nanofiber scaffold, where arrows indicate sebaceous glands (se), hair follicles (hf), hair roots (hr), and subcutaneous fat layer (s). (D) Photomicrographs of Masson's trichrome-stained nanofiber scaffold. (E) (a) Average width, (b) thickness, and (c) epidermal thickness of scars.

FIG. 9 is an SEM micrograph of (A) dECM, (B) PEU4:6 nanofiber mats and (C) cross-linked dECM nanofiber mats exposed to an open environment.

FIG. 10 shows the antibacterial activity of nanofiber scaffolds against P. aeruginosa and K. pneumoniae: Photographs of agar plates showing (A) the inhibition zone against bacterial pathogens and (B) the appearance and disappearance of bacteria incubated with different types of nanofiber scaffolds.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, the present disclosure will be described in more detail. However, the present disclosure may be implemented in many different forms, the present disclosure is not limited by the example embodiments described herein, and the present disclosure is defined only by the claims to be described later.

The terms used herein are presented for the description of the specific embodiments but are not intended to limit the present disclosure. In the entire specification, when a certain portion “comprises or includes” a certain component, this indicates that the other components are not excluded and may be further included unless specially described otherwise.

An example embodiment of the present disclosure relates to a nanofiber scaffold including polycaprolactone (poly(ε-caprolactone; PCL), decellularized extracellular matrix (dECM) and usnic acid (UA).

The nanofiber scaffold may be in the form of a nanofiber of the decellularized extracellular matrix and polycaprolactone containing usnic acid, but is not limited thereto.

The nanofiber scaffold may be prepared by electrospinning, but is not limited thereto.

The nanofiber scaffold may include the polycaprolactone and the decellularized extracellular matrix in a weight ratio of 8 to 2:2 to 8, for example, the polycaprolactone and the decellularized extracellular matrix in a volume ratio of 8:2, 6:4, 4:6 or 2:8, but is not limited thereto.

The nanofiber scaffold may be cross-linked, but is not limited thereto.

The nanofiber scaffold is prepared by a method including: mixing polycaprolactone, decellularized extracellular matrix, and usnic acid (phase 1); preparing a nanofiber mat by electrospinning the mixture (phase 2); cross-linking the nanofiber mat (phase 3); and freeze-drying the cross-linked nanofiber mat (phase 4), but is not limited thereto.

Hereinafter, the nanofiber scaffold of an example embodiment of the present disclosure will be described based on the preparation method of the nanofiber scaffold of an example embodiment of the present disclosure.

First, the preparation method of the nanofiber scaffold of an example embodiment of the present disclosure includes mixing polycaprolactone, decellularized extracellular matrix, and usnic acid (phase 1).

In phase 1, the polycaprolactone and the decellularized extracellular matrix may be mixed in a weight ratio of 8 to 2:2 to 8, for example, the polycaprolactone and the decellularized extracellular matrix may be mixed in a volume ratio of 8:2, 6:4, 4:6 or 2:8, but is not limited thereto.

In addition, the preparation method of the nanofiber scaffold of an example embodiment of the present disclosure includes: preparing a nanofiber mat by electrospinning the mixture (phase 2); cross-linking the nanofiber mat (phase 3); and freeze-drying the cross-linked nanofiber mat (phase 4).

In phase 2, the electrospinning may be performed using a technique commonly used in the pertinent field, but is not limited thereto.

In phase 3, the nanofiber mat may be immersed in EDC and NHS in ethanol to cross-link, but is not limited thereto.

In addition, an example embodiment of the present disclosure relates to a wound dressing including the nanofiber scaffold of an example embodiment of the present disclosure.

### EXAMPLES

### Material Preparation

Pure PCL, usnic acid (UA), 1-Ethyl-3-(3-dimethylaminopropyl) carbodiimide (EDC), N-hydroxysuccinimide (NHS), fluorescein diacetate (FDA), propidium iodide (PI), 3-(4,5-dimethylthiazol-2-yl)-2,5-diphenyltetrazolium bromide) (MTT), and 1, 1,1,3,3,3-hexafluoro-2-propanol (HFIP) were purchased from Sigma-Aldrich (USA). Dulbecco's minimum Eagle's medium (DMEM), fetal bovine serum (FBS), penicillin/streptomycin and trypsin (250 U/mg) were purchased from GIBCO © (Invitrogen Corporation, USA). Potato dextrose broth (PDB), tryptic soy broth (TSB), and brain heart infusion (BHI) were purchased from Difco Laboratory Inc. (Detroit, MI, USA). All other chemicals and solvents used in example embodiments of the present disclosure were of analytical grade and the water was deionized.

### Example 1

Preparation and Isolation of dECM

Decellularized extracellular matrix (dECM) was extracted from pig skin. First, the fat adhered to the lower side portion of the skin was physically removed and washed with distilled water. Then, the skin was finely cut and put in 0.25% of Trypsin-EDTA for 6 hours at 37° C. After incubation, debris was removed from the pig skin, washed with PBS (phosphate-buffered saline), and immersed in PBS containing 1% of Triton X-100 and 25 mM of EDTA for 24 hours. The pig skin was then washed with PBS for 24 hours at 4° C. and sterilized with 0.1% of peracetic acid in 4% of EtOH. Then, the pig skin was washed several times with distilled water, lyophilized, and dissolved in 1 M acetic acid containing pepsin for 7 days. The pH of the mixture was adjusted to 7.4 using NaOH solution under cold conditions, lyophilized again, and dissolved in HFIP for the preparation of nanofiber scaffolds.

### Preparation of Composite Nanofiber Mats

10% (w/v) of dECM and 10% (w/v) of PCL were dissolved in HFIP for Use Gap Code preparation, and PCL and dECM were mixed in different mass ratios (100:0, 80:20, 40:60, 60:40, 20:80, and 0:100) in the presence of usnic acid (UA) (3 mg/mL). The solution was electrospun through a 22-gauge needle into a stainless steel rotating collector wrapped in aluminum foil using a syringe pump (KD Scientific Inc., Holliston, MA, USA) with a high voltage power supplier under different operating conditions (Table 1). The nanofiber mat was completely immersed in pure ethanol containing EDC (20 mM) and NHS (5 mM) for 30 minutes, washed with pure ethanol, and lyophilized.

TABLE 1

| | Composition | UA | Flow | Volt- | Dis- | |
|---|---|---|---|---|---|---|
| Scaffold | (PCL [symbol] dECM) | (mg/ | rate | age | tance | Collector |
| name | (%) | mL) | (mL/h) | (kV) | (cm) | (RPM) |
| PCL | 100:0 | 0 | 2 | 9.5 | 10 | 200 |
| PCLU | 100:0 | 3 | 2 | 9.5 | 10 | 200 |
| PEU2:8 | 20:80 | 3 | 2 | 9.7 | 10 | 200 |
| PEU4:6 | 40:60 | 3 | 2 | 9.7 | 10 | 200 |
| PEU6:4 | 60:40 | 3 | 2 | 10 | 10 | 200 |
| PEU8:2 | 80:20 | 3 | 2 | 10.5 | 10 | 200 |

[symbol]

indicates data missing or illegible when filed

### Experimental Example 1

### Scanning Electron Microscopy (SEM)

The surface morphology of the nanofiber scaffold was analyzed using SEM. The electrospun composite nanofiber scaffold was sputter-coated with a silver layer using Emi-Tech K500X and visualized by SEM (FE-SEM, Hitachi S-2700, Japan) at an accelerating voltage of 5 kV. The average fiber diameter was analyzed with image analysis software (ImageJ Wayne, Rasband). In addition, the pore size distribution of the cross-linked scaffold was evaluated with image analysis software (ImageJ Wayne, Rasband) and the porosity was studied using the liquid-phase displacement method.

### Chemical Analysis by FTIR

Fourier-transform infrared spectrometer (FTIR) analysis was used for chemical characterization of pure UA and nanofiber dECM, PCL, PCLU, PEU 2:8, PEU 4:6, PEU 6:4, and PEU8:2 samples. All spectra were recorded at 4 cm intervals in the frequency range of 4000 to 650 cm¹ at room temperature using an FTIR (JASCO (FT-4100), Tokyo, Japan) spectrometer.

### X-ray Diffraction Analysis (XRD)

Pure UA, PCL, and composite nanofiber scaffolds were characterized by X-ray diffraction analysis (Philips X'pert MPD diffractometer, Eindhoven, Netherland) in the range of diffraction angles (20) from 10 to 600 at a scanning rate of 6°/min with CuKα radiation generated at 36 kV and 200 mA.

### Thermal Properties Analysis

Thermal properties of pure UA, PCL, and composite nanofiber scaffolds were measured at a constant heating rate of 10° C./min under nitrogen gas in the temperature range of 50 to 700° C. using a thermogravimetric (TGA) Pyris 1 TGA analyzer (Perkin Elmer TGA-7, Waltham, MA, USA). Melting properties were analyzed using differential scanning calorimetry (DSC) (Perkin Elmer, Diamond, Waltham, MA, USA) at a constant heating rate of 10° C./min under an argon gas flow of 50 mL/min in the temperature range from room temperature to 250° C.

### Mechanical Properties of Composite Scaffold

The tensile strength of the prepared nanofiber scaffold was identified using a universal tensile machine (LR5K Plus, Lloyd Instruments). A rectangular strip (15 mm×5 mm×T; T is the thickness) was placed and a crosshead speed of 1 mm/min was applied at room temperature. Three independent samples from each group were used for analysis.

### Contact Angle of Scaffold

The nanofiber scaffold was placed horizontally on a flat surface, and a droplet of DMEM (FBS free) was placed at room temperature. It was photographed with a digital camera and the contact angle was analyzed with image analysis software (ImageJ Wayne, Rasband) using three independent photographs per group.

### Swelling Properties of Composite Scaffolds

The prepared nanofiber scaffold was immersed in 1×PBS (pH 7.4) at 37° C. until equilibrium in an expansion state was reached. The nanofiber scaffolds were collected at regular time intervals, excess PBS and surface moisture were removed with blotting paper, and left at room temperature for 1 minute. Then, the equilibrium expansion ratio was calculated according to the following equation. In the equation below, W_{d} and Wₛ are dry weight and expanded weight, respectively.

Swelling (%)=[(Wₛ−W_{d})/W_{d}]×100

### In Vitro Degradation Properties

The prepared nanofiber scaffold was immersed in 1×PBS (pH 7.4) at 37° C. At regular intervals, the remaining samples were taken out, washed with deionized water and lyophilized. Weight loss was calculated according to the equation below. In the equation, Wᵢ and Wₜ are the initial dry weight and the weight at different times, respectively.

Degradation (%)=[(Wᵢ−Wₜ)/Wᵢ]×100

### In Vitro Release Properties

The release properties of usnic acid (UA) were identified. The prepared nanofiber scaffold was immersed in 1×PBS (pH 7.4), and the release medium was withdrawn at set time intervals and analyzed with a microplate reader (Gen 5™ ELISA BioTek, USA) at 280 nm. The released UA was calculated according to the equation below. In the equation, Wᵢ and Wₜ mean the initial amount of UA in the scaffold and the amount released at a specific time, respectively.

Release (%)=[(Wᵢ−Wₜ)/Wᵢ]×100

### Cell Culture and Cytotoxicity

Cell culture studies were conducted using HDF fibroblasts and HaCaT keratinocytes in DMEM supplemented with 10% of FBS, 100 units/mL of penicillin, and 0.1 mg/mL of streptomycin at 37° C. in a humidified 5% CO₂ atmosphere.

One hour prior to transfer to a 48-well microplate, the nanofiber scaffold was punched using an 8 mm diameter biopsy punch and exposed to UV irradiation. Cytocompatibility, cell adhesion, and proliferation on scaffolds were studied using the MTT assay at 1 and 5 days post seeding. First, 1×10⁴ cells were placed on an 8 mm scaffold and incubated in a humid environment at 37° C. On days 1 and 5 after seeding, cell-cultured nanofiber scaffolds were washed using 1×PBS (pH 7.4) and incubated in DMEM supplemented with MTT (1 mg/mL) for 3 hours in the same environment. The incubated solution was then aspirated and re-incubated with DMSO for 30 minutes at 37° C. The absorbance of the solution was measured at 540 nm using a microplate reader (Gen 5™ ELISA BioTek, USA).

### Fluorescence Assay

To study cytotoxicity, cell adhesion, proliferation, and migration on nanofiber scaffolds, live/dead cell staining was performed using FDA (5 μg/mL) and PI (20 μg/mL) in medium without FBS for 15 minutes at 37° C. on day 1 and day 5 after seeding the cells onto the nanofiber scaffold. After staining the cells with FDA and PI, the solution was aspirated and washed twice with 1×PBS (pH 7.4). Green fluorescence of live cells and red fluorescence of dead cells were detected using a fluorescence microscope (Leica DMI300B Microsystems, Wetzlar, Germany).

### Antibacterial Activity

Bacterial cultures of Pseudomonas aeruginosa (PAO1 KCTC 1637), Staphylococcus aureus (KCTC 1916), Cutibacterium acnes (KCTC 3314), and Klebsiella pneumoniae (ATCC 4352) were purchased from Korean Collection for Type Cultures (KCTC, Daejeon, Korea). Streptococcus mutans (KCCM 40105), Staphylococcus epidermidis (KCCM 40003), and fungal strain Candida albicans (KCCM 11282) were purchased from Korean Culture Center of Microorganisms (KCCM, Sudaemun Seoul, Korea). The growth medium for the culture of C. albicans is PDB supplemented with 5% of glucose, and the agar medium is PDA. P. aeruginosa, S. aureus, K. pneumoniae, and S. epidermidis used TSB as the growth medium and tryptic soy agar (TSA) as the agar medium. C. acnes was grown and cultured in BHI supplemented with 1% of glucose and BHA (brain heart infusion agar) in a CO₂ incubator (NAPCO 5400; General Laboratory Supply, Pasadena, USA) in a 10% CO₂ humid atmosphere. The growth temperature of all bacterial strains and C. albicans was 37° C.

### Antibacterial and Antifungal Activity

The antibacterial activity of nanofiber scaffolds against bacterial and fungal pathogens was identified by solid agar labeling and disk diffusion in liquid growth media. Cultures of bacterial pathogens and C. albicans grown overnight were diluted to an equivalent OD₆₀₀ value of 0.05 (same as the Mcfarland standard for bacterial inoculum), and were dispensed (100 μL) onto the labels of TSA (for bacterial culture) and PDA (for fungal cell culture) plates using a cotton swab. Spreading of the cell culture was performed by forming regular bacterial cultures on agar plates. An 8 mm diameter of nanofiber scaffold was placed on the surface of an agar plate including a bacterial or fungal cell culture and incubated at 37° C. for 24 hours. After incubation, inhibition of diameter was measured.

Two nanofiber scaffolds (8 mm diameter) were placed on a 24-well microplate containing bacterial or fungal cell cultures. Bacterial cell cultures were incubated for 24 hours and C. albicans cell cultures were incubated for 48 hours while shaking the microplate at 150 rpm at 37° C. C. acnes cell cultures in BHI supplemented with glucose (1%) on a 24-well microplate were incubated with nanofiber scaffolds in a CO₂ incubator (NAPCO 5400; General Laboratory Supply, Pasadena, USA) at 37° C. and 10% CO₂ humid atmosphere for 48 hours under anoxic conditions. After incubation, the cell culture (100 μL) was serially diluted (10⁻⁸ dilutions) with TSB (for bacterial culture) and PDB (C. albicans). After spreading the diluted cell culture (100 μL) on TSA and PDA agar plates, bacterial cells were grown by incubation at 37° C. for 24 hours and C. albicans cells for 48 hours. C. acnes bacterial cell cultures were spread-plated on BHA and incubated for 24 hours at 37° C. under anoxic conditions. The total number of colonies on each agar plate was counted to measure colony-forming units (CFU).

### Anti-Biofilm Assay

Biofilm inhibitory properties of the nanofiber scaffold against P. aeruginosa and K. pneumoniae were identified. An 8 mm diameter of nanofiber scaffold was used to identify the biofilm inhibitory activity. The biofilm inhibitory activity of the nanofiber scaffold was identified by measuring the CFU of cells adhered to the surface. The nanofiber scaffold was placed at the bottom of a 24-well microtiter plate and the diluted cell culture was dispensed (1:100 dilution in its respective growth medium). Microtiter plates were incubated for 24 hours at 37° C. under static conditions. After incubation, the free-floating suspension cell culture was discarded and the nanofiber scaffold was washed 3 times with sterile growth medium and transferred to a centrifuge tube (1.5 mL). The centrifuge tube containing the nanofiber scaffold was resuspended with 500 μL of TSB (bacteria) and PDB (C. albicans). The tube was vortexed for 5 minutes and then sonicated for 10 minutes. The cell suspension (100 μL) from the centrifuge tube was diluted up to 108 dilutions with each sterile growth medium. Diluted cell cultures (100 μL) were spread-plated on agar plates and incubated at 37° C. for 24 hours. The percent inhibition of the biofilm was calculated according to the equation below. In the following equation, N_{c} represents the average number of microbial colonies in the control group, and Nₛᵢ represents the average number of microbial colonies in the experimental group.

Inhibition (%)=(N_{c}−Nₛᵢ)/N_{c}×100

### In Vivo Wound Healing Model

The in vivo wound healing properties of nanofiber scaffolds were evaluated using 8-week-old normal male ICR mice (6 mice per group) weighing 30 g and acclimatized for 7 days under a controlled environment (relative humidity: 40 to 70%, temperature: 20 to 24° C.). All mice were anesthetized in a chamber containing 20% of isoflurane in isopropanol for 20 minutes and masked throughout the surgery. After shaving the hair and disinfecting with povidone-iodine and 70% of ethanol, a full-thickness 2.25 cm² square excisional wound was created on the skin of the back. The prepared PCL, PCLU, PCL:dECM 4:6 (PE 4:6), and PEU 4:6 nanofiber scaffolds were positioned using sutures. After surgery, the wound areas of all experimental groups were photographed on days 0, 7, 14, and 21 with a digital camera, and wound closure was measured with image analysis software (ImageJ Wayne, Rasband) and plotted as a relative percentage of initial wound size. For histomorphological evaluation, mice were sacrificed on day 21 after surgery, and wound sites and tissues therearound were collected.

### Histomorphometric Analysis

The collected tissue samples were fixed for 1 day using a 10% of formalin solution, dehydrated using alcohol and xylene successively, and wax infiltrated using a tissue processor (Leica TP1020, Leica Biosystems, Nussloch, Germany). Embedded tissues (Leica EG1160, Leica Biosystems, Nussloch, Germany) were sectioned at 5 μm, mounted on glass slides, dewaxed with xylene and then dehydrated with alcohol and then water. Nuclei were stained with hematoxylin, excess hematoxylin was washed away with water and ECM and cytoplasm were counterstained with eosin. The stained tissue was dehydrated by applying grading alcohol up to 100%, washed in xylene, and placed on a cover-slip using a mounting medium.

In addition, collagen deposition was evaluated by staining the dissected tissues with Masson's trichrome staining (Masson's Trichrome stain kit; KTMRT, American MasterTech, Lodi, CA, USA). First, deparaffinized incisions were immersed in Bouin's fluid preheated at 60° C. for 60 minutes, washed in running tap water, stained with working Weigert's hematoxylin for 5 minutes, and washed with tap water. Then, the deparaffinized incisions were impregnated in Biebrich scarlet for 15 minutes, washed with tap water, incubated with phosphotungstic/phosphomolybdic acid for 10 minutes, impregnated in aniline blue for 5 minutes, washed with distilled water, transferred to 1% of acetic acid, dehydrated with absolute ethanol, and placed on a cover-slip using a mounting medium. Finally, the stained incisions were observed using an optical microscope (Leica Microsystems UK Ltd, Milton Keynes, UK) and representative photographs were taken.

### Statistical Analysis

All quantitative data are presented as the mean±standard deviation (SD) from three independent experiments performed using fresh reagents. Significant differences between groups were evaluated using one-way ANOVA according to Duncan's test using GraphPad Prism 7.0 (GraphPad Software Inc., San Diego, CA, USA). *p<0.05 and **p<0.01 were selected as having statistical significance compared to control.

### Experimental Example 2

### Morphological Characterization of Electrospun Nanofibers

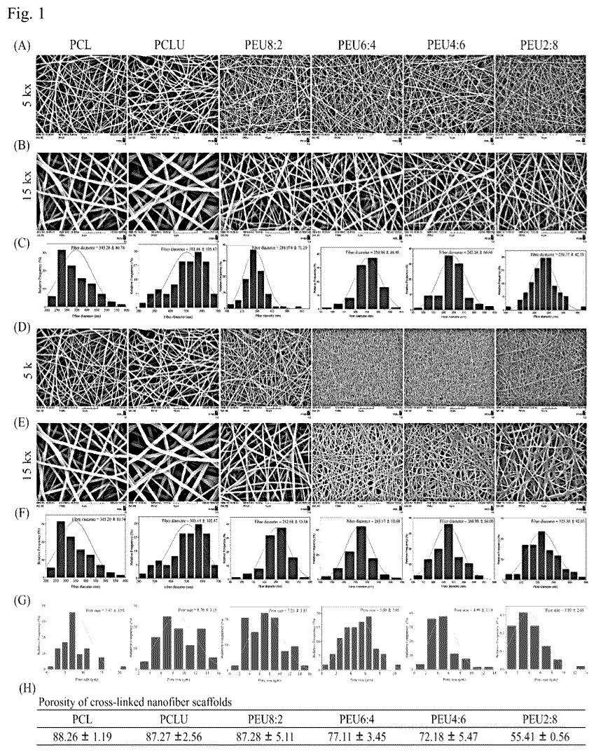

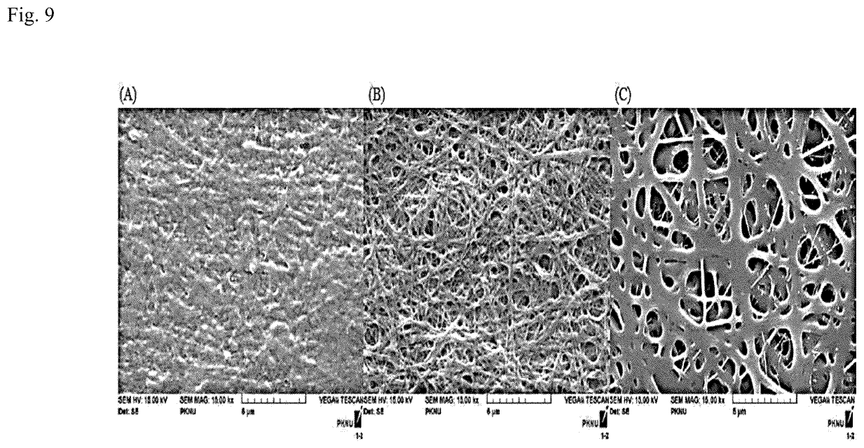

Bead-free, randomly arranged, smoothly and homogeneously distributed nanofiber structures were obtained by controlling operating conditions such as voltage, distance between drum collector and needle, and flow rate of polymer solution, regardless of polymer solution concentration. (A), (B) and (C) of FIG. 1 show that pure PCL and PCLU nanofibers have larger average diameters than PEU nanofibers (345.20±80.74 nm and 503.44±105.47 nm, respectively). The composite PEU nanofibers showed different average fiber diameters of 284.94±71.29 nm, 254.88±46.98 nm, 242.26±66.66 nm, and 230.77±42.10 nm, respectively, depending on the combination of PCL and dECM (dECM combinations of 20%, 40%, 60%, 80%, and 100%). An increase in dECM decreased the viscosity of the polymer mixture, so an increase in dECM content decreased the fiber diameter. Since dECM has high solubility in water, high sensitivity to humidity (SEM photomicrographs of dECM and a PEU4:6 nanofibers taken in open environment, FIG. 9), and immediate degradability, only a small amount of 100% ethanol-based EDC/NHS for a very short time was able to provide minimally effective surface cross-linking in UA-blended polymer solutions. (D), (E) and (F) of FIG. 1 show morphological changes in the nanofibrous structure of the scaffold after cross-linking. Fiber morphology remained unchanged at relatively low dECM content (PEU8:2), but showed minimal fusion of fiber connections. PEU scaffolds with high content of dECM showed morphological changes, but dECM-only nanofibers showed greater morphological changes with both connected and fused fibers together even when exposed only to a small amount of cross-linking solution for a short period of time ((C) of FIG. 9). It was identified whether the pore size of the cross-linked PEU scaffold ((F) of FIG. 1) decreased as the dECM content increased, with PEU 2:8 having a smaller pore size (3.89±2.52 μm) and PEU 4:6 and PEU 6:4 having an average pore size (4.95±2.19 μm and 5.00±2.05 μm, respectively). These pore sizes were large enough to provide skin cells. Moreover, PEU 2:8 had a fairly low porosity, and both PEU 4:6 and PEU 6:4 had a porosity suitable for receiving cells ((G) of FIG. 1). Taken together, these results indicate that PEU 4:6 and PEU 6:4 have the most ECM-like properties because of their fibrous and porous structures, which facilitates their application in cell activity and skin tissue regeneration.

### FTIR Spectroscopy

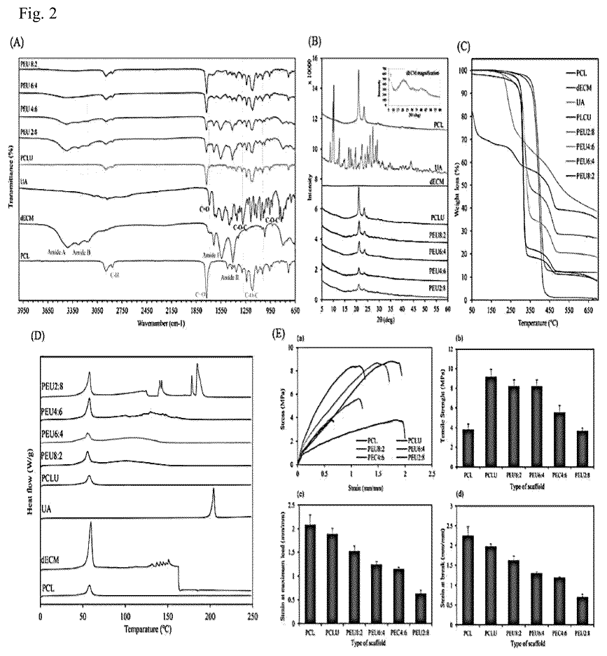

The FTIR spectra of the PCL, dECM, UA, and cross-linked composite PEU nanofiber scaffolds, and shifts in the characteristic absorption bands are shown in (A) of FIG. 2. The PCL spectra showed a characteristic peak at 2938 cm⁻¹ and 2862 cm⁻¹ (asymmetric and symmetric —CH stretching, respectively), 1721 cm⁻¹ (carbonyl C═O stretching), and 1237 cm⁻¹ and 1165 cm⁻¹ (asymmetric and symmetric C—O—C stretching, respectively). The IR spectra of dECM showed five characteristic bands at 3406 cm⁻¹, 3154 cm⁻¹, 1635 cm⁻¹, 1522 cm⁻¹, and 1233 cm⁻¹ corresponding to amide A (N—H stretching vibration), amide B (H—H stretching vibration), amide I (C═O stretching vibration coupled to N—H vibration), amide II (N—H bending vibration), and amide III (N—H deformation and C—N stretching), respectively. These peaks identified in the FTIR spectrum almost matched those in the collagen FTIR spectrum (T. Riaz et al., Applied Spectroscopy Reviews, 53 (2018) 703-746). UA spectra showed characteristic absorption bands at 1687 cm⁻¹ (C═O stretching coupled to cyclic ketone groups), 1632 cm⁻¹ (non-aromatic ketone), and 1282 cm⁻¹ and 1042 cm⁻¹ (antisymmetric and symmetric C—O—C aryl alkyl ether modes, respectively). The UA-induced peaks in the nanofiber PCLU scaffolds were not seen because the amount of UA present in PCLU was significantly lower than in PCL, which led to the hypothesis of forming hydrogen bonds, consistent with previous studies where the composite polymer film reduced the intensity of the peak related to a polymer and made the peak of UA disappear. However, previous studies of the present inventors demonstrated that the peaks related to UA of UA-chitosan nanoparticles remained visible, and that the sharpness of chitosan peaks decreased due to the use of a larger proportion of UA depending on the chitosan in the nanoparticles, and suggested that bond formation as well as material concentration and representative vibration peak overlapping might affect the appearance and disappearance of vibrational peaks. For the composite PEU nanofiber scaffold, the intensity of the absorbance peak related to dECM decreased with the decrease of the dECM content in the composite, while the peak related to UA disappeared with higher intensities of PCL and dECM, which is also consistent with the previous hypothesis (C. Pagano et al., Colloids and Surfaces B: Biointerfaces, 178 (2019) 488-499). However, the intensity of the absorbance peaks related to dECM decreased as the dECM content in the composite nanofiber PEU scaffold decreased, while the peaks related to UA disappeared due to the high intensities of PCL and dECM. Residual peaks corresponding to the antisymmetric C—O—C stretching vibration in all composite PEU nanofiber scaffolds representing PCL, dECM and UA were well distributed within the composite PEU nanofiber scaffold even after weak cross-linking treatment.

### X-ray Diffraction Analysis

(B) of FIG. 2 shows the X-ray diffraction patterns of pure UA and electrospun PCL, dECM, PCLU, and composite PEU scaffolds. The PCL nanofibers showed a sharp diffraction peak at 20=21° and a relatively low-intensity peak at 23°, which correspond to the (110) and (200) reflections of polyethylene-like structures with orthorhombic unit cell parameters, identifying that PCL has semi-crystalline properties. dECM is a complex structure composed mainly of collagen, and is an amorphous material with a broad peak at 2θ=˜22° without a specific sharp peak in the X-ray diffraction pattern. The UA powder showed characteristic sharp peaks similar to previous studies, indicating a unique crystalline nature. The cross-linked composite nanofiber PEU scaffold had only two characteristic PCL peaks at 2θ=21° and a low intensity peak at 23°, and the intensity decreased as the dECM content in the scaffold increased, demonstrating that the decrease in the degree of crystallinity of the composite nanofiber scaffold was attributed to the introduction of amorphous dECM. These results are consistent with previous study results in electrospun PCL-collagen nanofibers (D. Chen et al., International journal of nanomedicine, 14 (2019) 2127). There were no characteristic UA diffraction peaks in the composite scaffolds, but minimal diffraction due to the limited availability of UA in the composite scaffolds.

### Thermal Behavior Analysis

TGA ((C) of FIG. 2) was performed to evaluate the thermal degradation behavior of PCL, dECM, UA, and composite PEU nanofiber scaffolds. Similar to previous studies, PCL showed a suitable single-shot thermal degradation profile (90% weight loss) with no char residues starting at ˜370° C. and ending at ˜470° C. dECM showed a two-phase degradation: ˜40% weight loss due to loss of bound water up to ˜200° C. and nearly 30% weight loss from ˜200° C. to ˜500° C. as a result of breakage of protein chains and rupture of peptide bonds. UA showed a two-stage weight loss: Weight loss at a low rate while retaining the most char residue after ˜30% weight loss up to 280° C. In thermal degradation profiles, the residue percentages at 550° C. of pure PCL, dECM, UA, and composite PEU2:8, PEU4:6, PEU6:4, and PEU8:2 nanofiber scaffolds were 0%, 35%, 38%, 26%, 18%, 12%, and 8%, respectively, indicating that the composite PEU scaffolds are among the pure materials. Accordingly, the degradation profile of the composite PEU scaffold is a combination of the thermal profile of the pure materials. The increase of dECM in a composite PEU system increased thermal stability, and both PEU 4:6 and PEU8:2 showed the highest thermal stability.

### DSC Analysis

The miscibility and homogeneity of the dECM, PCL, and UA nanofiber blends were identified by DSC analysis. Theoretically, perfectly miscible and homogeneous complexes form a single-phase transition instead of the formation of mixed phases and other phase transitions of each pure material used in the complex. The results ((D) of FIG. 2) showed that PCL, dECM, and UA, which had melting points of about 59° C., 60° C., and 206° C., respectively, started to change from solid to liquid at ˜41° C., −41° C., and 194° C., respectively. The composite PEU 2:8, PEU 4:6, PEU 6:4, and PEU8:2 nanofibers electrospun from the blend solution in HFIP showed single endothermic peaks at ˜57° C., −57° C., 58° C., and 58° C., respectively, all of which were almost similar to the endothermic peaks of pure PCL and dECM. The peak corresponding to the melting point of UA disappeared, probably because very little UA was present in the composite fiber. These results suggest that all composite PEU blends in HFIP do not undergo phase separation during the coagulation process in the electrospinning stage. Taken together, it was demonstrated that PEU 4:6 and PEU 2:8 possessed additional dECM properties and were able to be used for wound healing.

### Mechanical Properties

The mechanical integrity of nanofiber scaffolds is essential for their application in tissue regeneration as it is important to supply basic biomechanical fitness to cells before tissue regeneration. The mechanical properties of nanofiber PCL, PCLU, and PEU scaffolds with different dECM contents were analyzed, and typical tensile stress-strain curves ((E)(a) of FIG. 2), tensile strength ((E)(b) of FIG. 2), strain at maximum weight ((E)(c) of FIG. 2), and strain at maximum extension ((E)(d) of FIG. 2) were plotted. In (E) a and b of FIG. 2, the nanofiber scaffold composed of pure PCL had the highest extensibility and the lowest tensile strength. Conversely, the PEU composite nanofiber scaffold (PEU8:2) including the highest PCL composition had the highest tensile strength (8.2±0.6 MPa) and extensibility (1.5±0.1 mm/mm), while subsequent composite nanofiber PEU scaffolds had reduced average tensile strengths of 8.2±0.6 MPa, 5.5±0.7 MPa, and 3.7±0.2 MPa and an extensibility of 1.2±0.0 mm/mm, 1.1±0.0 mm/mm, and 0.6±0.1 mm/mm with increasing dECM content, indicating that dECM had low mechanical stability similar to collagen. The scaffold with the highest dECM content (PEU2:8) had the lowest mechanical stability, indicating that PEU 2:8 may not provide suitable mechanical strength for skin tissue regeneration.

### Water Contact Angle

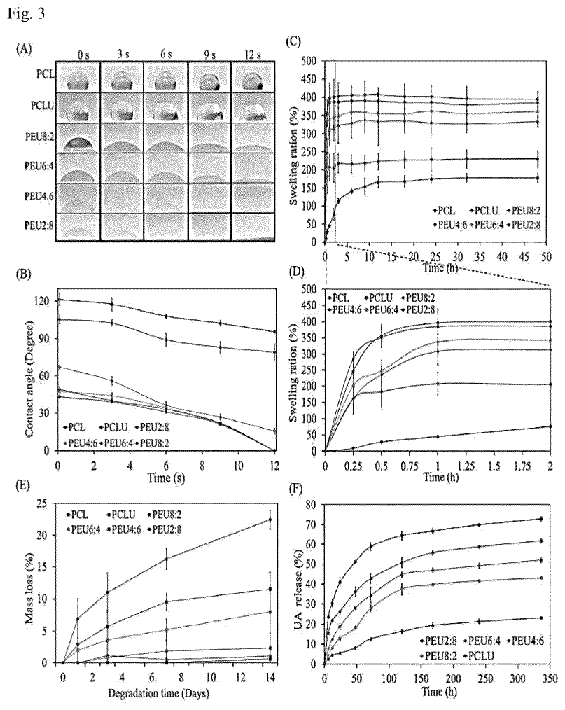

Since the hydrophilic nature of composite nanofiber scaffolds is one of the most important properties for water absorption, the wettability of PCL, PCLU, and composite PEU nanofiber scaffolds was identified using water contact angle. (A) and (B) of FIG. 3 show photographs of water contact angles of PCL, PCLU, and composite PEU nanofiber scaffolds and contact angles over time. Although the surface structure changed as the dECM content increased, the nanofiber PEU8:2, PEU 6:4, PEU 4:6, and PEU 2:8 scaffolds exhibited water contact angles of 67.5°±2.9°, 47.4°±4.0°, 49.8°±4.5°, and 43.1°±4.1°, respectively, at 0 second, lower than that of the pure PCL nanofiber scaffold (122.1±4.1°). The water contact angle rapidly decreased to zero, which indicates that the composite PEU nanofibers, especially PEU 2:8, PEU 4:6, and PEU 6:4, had high hydrophilic natures due to their high dECM content and highly interconnected pore structure. PEU 6:4, PEU 4:6, and PEU 2:8 also showed faster water permeability than PEU8:2, indicating that they have high infiltration.

### Swelling Properties

The swelling capacity of tissue-engineered skin substitutes is important to absorb exudates, which are main ingredients that stimulate cell migration and proliferation, while providing a moist environment at the wound site. The expansion ratios of PCL, PCLU, and all PEU composite nanofiber scaffolds are shown in (C) and (D) of FIG. 3. Composite PEU nanofiber scaffolds showed a higher swelling ratio (300% or more) compared to pure PCL and PCLU nanofiber scaffolds because the PEU composite scaffold was mainly composed of collagen and included dECM with high water absorption capacity. The swelling behavior of the composite PEU scaffolds reached a near-steady state within 2 hours, but the swelling ratio of the PEU scaffolds showed a tendency for the swelling capacity to increase with increasing dECM content. The swelling ratio of PCLU reached an early steady state within 2 hours, which is probably due to the regular pore distribution and changes in water contact properties by UA distributed in the scaffold, even though UA is a water-insoluble material. Conversely, the PEU nanofiber scaffolds, especially PEU4:6 and PEU2:8, had relatively high swelling properties and reached the steady swelling state early, mainly due to the molecular and structural properties of the protein-based polymers in dECM.

### In Vitro Degradation

The prepared nanofiber scaffolds also need to have suitable degradation properties that allow successful tissue regeneration and replacement of biological tissue with structures. Weighted analysis ((E) of FIG. 3) showed that the pure PCL and PCLU nanofiber scaffolds showed little degradation. However, their degradation rates increased with increasing dECM content. The PEU8:2 nanofiber scaffold showed the lowest degradation rate (2.3%±0.8%), and the PEU 2:8 showed the highest degradation rate more than 10 times that of the PEU8:2 scaffold. However, PEU 6:4 and PEU 4:6 showed moderate degradation rates. A high degradation rate is due to release of non-cross-linked dECM and surface degradation by the limited cross-linking used, while a low degradation rate indicates more entrapment within the PCL scaffold.

### In Vitro Release Properties

As can be seen in (F) of FIG. 3, the PCLU scaffold was observed to have the lowest release property, reaching 23.0±0.7% even on day 14, because the PCL had a dense crystalline structure and held the UA firmly. However, it was found that the PEU scaffold had a controlled release behavior due to the amount of dECM contained. The capacity of the PEU polymer matrix to degrade and swell, as well as drug diffusion from the fibers, is attributed to the release mechanism of UA. Initially, UA was initially released from all PEU scaffolds and then continuously. PEU 2:8 showed the highest initial release (40.7±2.4 on day 1), which was attributed to the loosening of the physical interaction between PCL and dECM and the high swelling and degradation of dECM. Conversely, PEU 4:6 and PEU 6: showed a more controlled and sustained release over 14 days, which is due to the formation of a low-decomposable synthetic-natural material with high stability.

### Antibacterial and Antifungal Activities

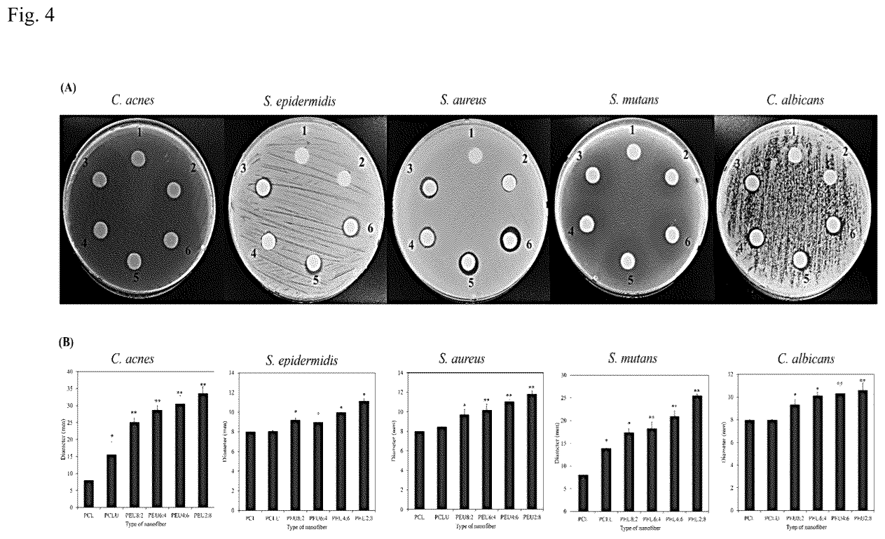

The disk diffusion assay results showed a clear zone in which all types of bacterial and fungal pathogens were inhibited in the presence of the UA-integrated scaffold, and the diameter of the inhibition zone increased as the dECM content in the nanofiber scaffold increased. Among the pathogens experimented, C. acnes and S. mutans showed larger inhibition zones on both UA-integrated PCLU and PEU scaffolds ((A) of FIG. 4), indicating that UA had good antibacterial activity. The diameter of the inhibition zone for C. acnes was 33.60±1.9 mm at the highest dECM concentration (PEU2:8), whereas the diameter of the inhibition zone for S. mutans was 25.47±0.3 mm. Compared with PCL (control) and PCLU (containing 0.3% of UA), the inhibition zone of PEU 2:8 nanofiber scaffold against C. acnes was 2- and 3-fold wider. Conversely, no inhibition zone was observed in PCL (without containing UA). As the dECM concentration increased, the antibacterial effect of the nanofiber scaffolds against S. epidermidis, S. aureus, and C. albicans increased. The diameter of the inhibition zone ranged from 1 to 3 mm compared to the control PCL scaffold, demonstrating increased solubility of UA. A representative photograph of an agar plate showing the inhibition zone of each nanofiber scaffold is shown in (A) of FIG. 4.

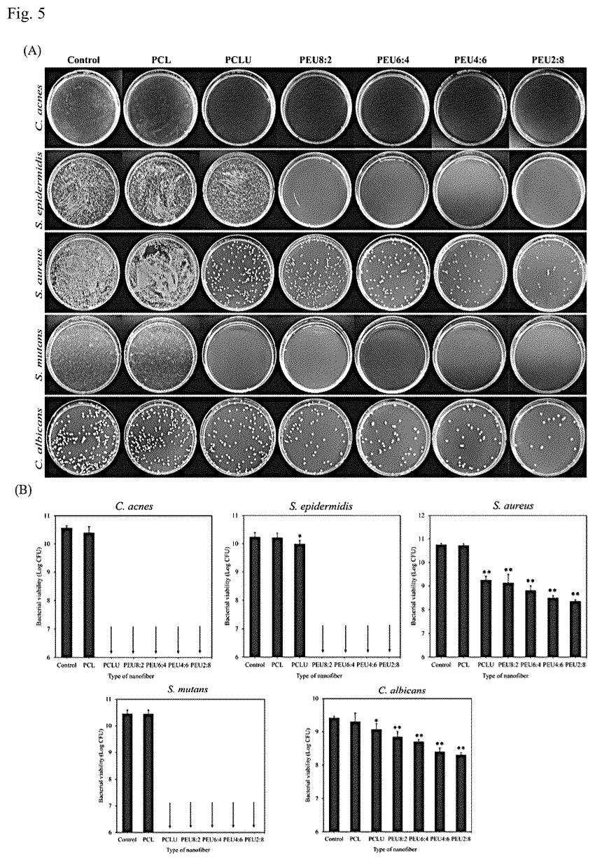

The antibacterial effect of the nanofiber scaffolds against pathogens was experimented in liquid broth medium and representative agar plates, showing the appearance and disappearance of bacterial and strain cell cultures ((A) of FIG. 5) as well as the number of CFUs ((B) of FIG. 5). All nanofiber PEU scaffolds showed complete bactericidal effect against pathogens. In particular, PCLU also completely sterilized pathogens, which proves that it has a high antibacterial effect even though UA is not significantly released. Similarly, the complete sterilization of S. epidermidis cells was also observed for all types of nanofiber scaffolds except for nanofiber scaffolds not containing dECM. S. aureus and C. albicans cells were not completely sterilized, but the cells were significantly sterilized in a dECM-dependent manner, with log CFU ranging from 0.34 to 1.1 (C. albicans) and 1.5 to 2.4 (S. aureus).

Based on the above results, it was concluded that the increased concentration of dECM showed a higher antibacterial effect against pathogens compared to the control group. The high antibacterial effect by high content of dECM suggests that the high content of dECM in nanofiber scaffolds results in high release properties accompanied by increased swelling, surface erosion and degradation. However, the antibacterial effect of PCLU clarifies the antibacterial effect of UA despite the minimal release and fiber degradation. Previous studies have shown that UA-loaded nanofibers, UA-encapsulated nanomaterials and hydrogels show antibacterial effects similar to those of the present disclosure.

### Anti-Biofilm Activity

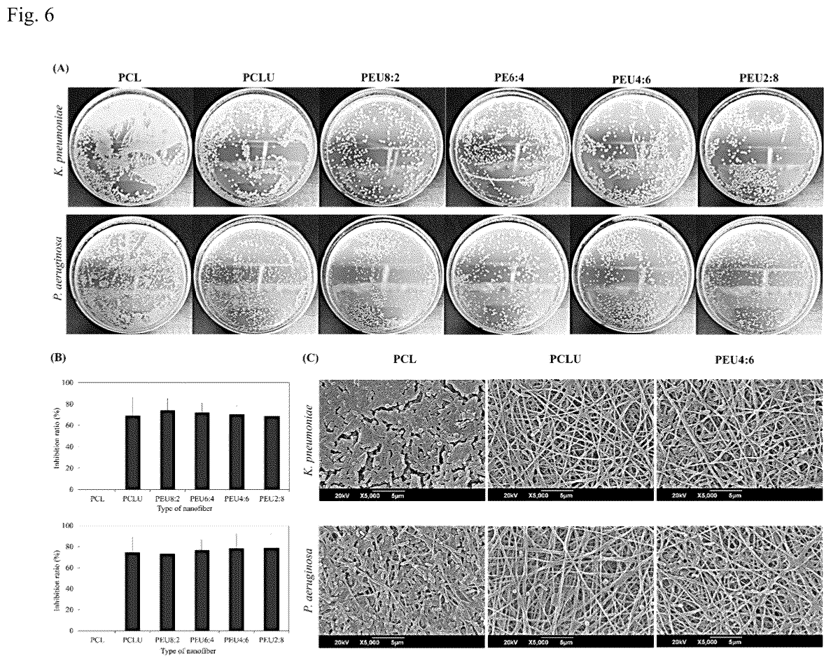

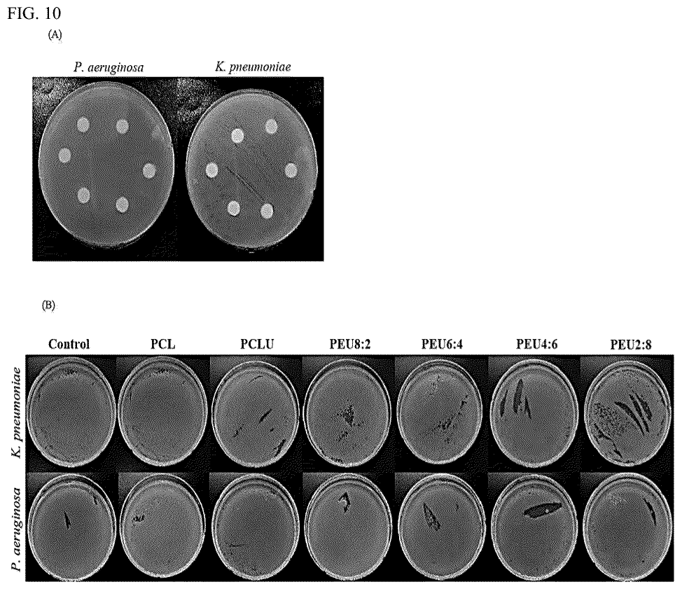

The formation of microbial biofilms on biological/non-biological surfaces, including medical products, is a major source of microbial infection and poses a serious threat to human health. A biofilm is made of a homogeneous group of microbial cells encapsulated in a self-produced extracellular polymeric material. Microbial biofilms act as barriers and protect against host immune responses, so they are one of the major obstacles to the treatment of infections and cause failure of antibacterial treatment. Biofilm formation by pathogenic microorganisms, such as those found at wound sites, is a major cause of wound infection treatment failure. In an example embodiment of the present disclosure, the inhibitory ability of the PEU nanofiber scaffold against biofilm formation of microbial pathogens related to burn wound infection, such as P. aeruginosa and K. pneumoniae (PMID: 26500905 and 18158583), was identified. It was identified through antibacterial assays that the nanofiber scaffolds had effective antibacterial and antifungal activities against C. acnes, S. aureus, S. epidermidis, and S. mutans. However, the nanofiber scaffold showed no antibacterial effect against K. pneumoniae and P. aeruginosa (FIG. 10). Accordingly, only K. pneumoniae and P. aeruginosa bacterial strains were used to test the anti-biofilm efficacy of nanofiber PEU scaffolds. As a result, compared to the control (PCL), all types of nanofiber scaffolds exhibited anti-biofilm properties ((A) and (B) of FIG. 6). All nanofiber scaffolds showed 60% to 68% and 70% to 80% biofilm inhibition rates against K. pneumoniae and P. aeruginosa, respectively. The biofilm structure was experimented using SEM to identify the biofilm inhibitory activity of the nanofiber scaffold. The SEM results showed that all nanofiber scaffolds had significantly fewer K. pneumoniae and P. aeruginosa cells adhered to the surface compared to the control scaffold ((C) of FIG. 6). Accordingly, from the above results, it was identified that the UA-integrated nanofiber scaffold had anti-biofilm properties against bacterial pathogens that mainly cause wound infections in humans.

### In Vitro Biocompatibility

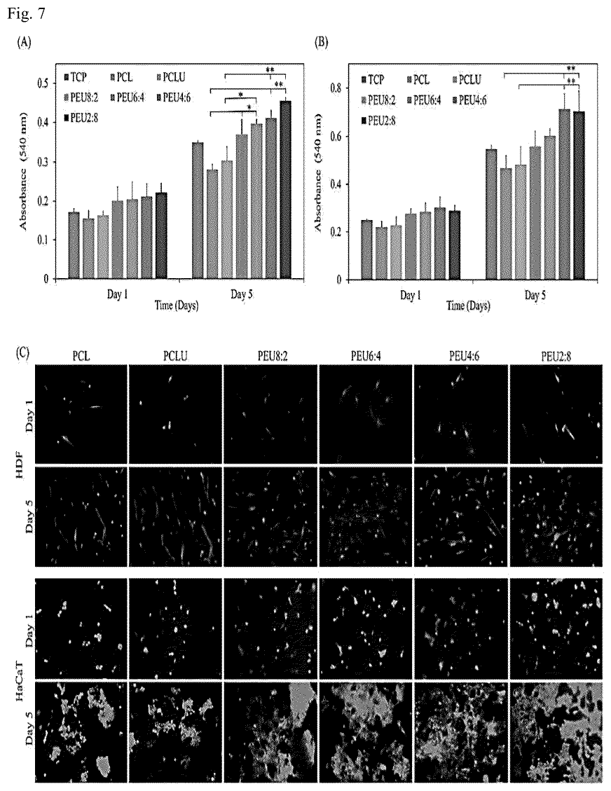

Cell viability and proliferation as a function of time on nanofiber scaffolds are indicators of the scaffold's cell compatibility and suitability for tissue engineering applications. Through the MTT assay, it was identified that the nanofiber scaffold satisfied the basic cell compatibility requirements for application in tissue engineering. Adhesion and proliferation of HDF and HaCaT cells at 1 and 5 days after culture on pure PCL, PCLU, and composite PEU nanofibers are shown in (A) and (B) of FIG. 7, respectively. At day 1, composite nanofiber PEU scaffolds showed higher cell counts than tissue culture plates (TCP) and pure PCL and PCLU nanofiber scaffolds, suggesting that dECM-integrated scaffolds provide native ECM-like structures and properties for cell adhesion and growth. This is because the cell adhesion motif present in the structural proteins of dECM promotes cell-ECM and cell-cell interactions. PCLU also showed slightly higher cell counts than PCL, which may be due to increased wettability (FIG. 3) and biological properties achieved by the presence of UA. UA alone was highly toxic to cells because UA immediately formed sharp crystals in the culture medium and causes cell damage, but became non-toxic when integrated with polymeric materials. At day 5, as the dECM composition of the composite PEU scaffolds increased, cell proliferation also significantly increased. This is because the increase in dECM content increased the possibility of integrin receptors and other physical and biochemical signals to promote cell adhesion, activated biological signals and cell-cell, cell-dECM, and cell-soluble molecular interactions within cells that cause cell survival, proliferation, and migration, and increased the solubility of UA.

The biocompatibility of the scaffold was further evaluated using FDA and PI staining for live/dead cell labeling. (C) of FIG. 7 shows microscopic images of cells cultured on nanofiber scaffolds on day 1 and day 5. One day after the HDF and HaCaT cells were dispensed, cells were adhered and spread all over the surface of all nanofiber scaffolds, but fewer cells were adhered to PCL than to PEU. The cell counts on the PEU scaffold increased with increasing dECM content. Increased cell counts on dECM-containing scaffolds were consistent with the MTT assay results, demonstrating that dECM promoted cell adhesion. After 5 days, the live cell density increased, but the number of dead cells according to the dECM content in the PEU scaffold was negligible, indicating that the nanofiber scaffold was not toxic to cells and cell proliferation and migration were promoted by various physical and biological signals provided by components of dECM, such as integrin receptors and collagen. Moreover, PCLU nanofibers showed more cell growth and proliferation than PCL nanofibers, indicating that UA promoted cell proliferation.

### Visual Observation of Wound Closure

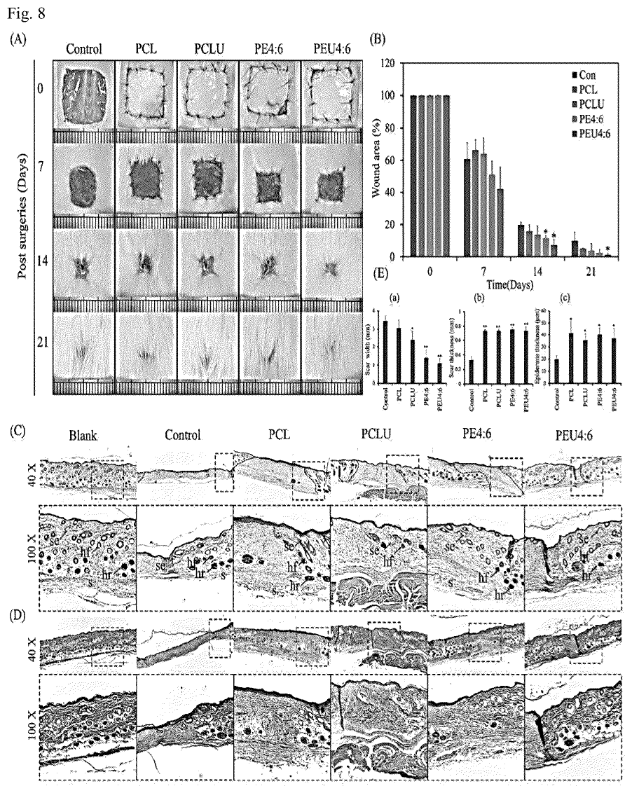

(A) and (B) of FIG. 8 show images and analysis of full-thickness wound sites after excision in different groups (in other words, untreated, pure PCL, PCLU, PE 4:6, and PEU 4:6 nanofiber scaffolds) at days 0, 7, 14, and 21. No animals showed any postoperative side effects or complications or died until sacrificed for histological evaluation. Regardless of nanofiber treatment, all wounds contracted gradually and fully recovered within 21 days after surgery, but the control group developed wider scars even over time. Wounds treated with PE 4:6 and PEU 4:6 closed faster than wounds treated with other substitutes, and wounds treated with PEU 4:6 showed the fastest wound closure. Compared to the control group (60.8%±10.0%, 19.8%±1.70%, and 9.9%±5.0%), the wound area of the PEU4:6-treated group at 7, 14, and 21 days was reduced by 42.1%±13.8%, 7.2%±3.4%, and 1.3%±1.0%, and the wound area of the PE4:6-treated group was reduced by 51.3%±8.2%, 11.6%±1.4%, and 2.7%±2.0%. PCLU-treated wounds also healed rapidly, but slower than PE 4:6-treated and PEU 4:6-treated groups, but faster than pure PCL-treated groups. When comparing UA-integrated nanofibers and non-integrated nanofibers, PEU 4:6 nanofibers showed faster wound closure, suggesting that UA increases wound healing as a marine-derived compound with excellent biological activity and antibacterial and wound healing properties. In line with these findings, direct topical administration and administration via other techniques may also accelerate wound healing. In addition, the dECM-integrated PE 4:6 group showed significantly faster wound closure compared to the control and PCL-treated groups. This is because skin-derived dECM is a complex source of not only polysaccharides such as glycosaminoglycans but proteins that increase and precisely control cellular functions such as cell adhesion, proliferation, differentiation, and migration such as collagen, laminin and fibronectin. Taken together, these results demonstrate that dECM in nanofiber scaffolds are useful for wound tissue regeneration and support UA to accelerate wound healing.

### Histological Analysis

To identify the influence of the composite nanofiber scaffold on skin tissue regeneration, a histomorphometric test was performed on the center of the wound site on day 21 after staining the wound with hematoxylin and eosin (H&E) staining ((C) of FIG. 8). All groups, including the control group, were completely re-epithelialized, although with varying degrees of residual scarring and remodeled tissue. Epithelialization of the control group was almost completed at 21 days after injury (a thickness of 20.00±2.94 μm), but the scar tissue was thinner (a thickness of 0.33±0.05 mm) and wider (a width of 3.45±0.25 mm) compared to the treatment group ((E) of FIG. 8). Wounds treated with PEU 4:6 showed faster scar tissue formation and granulation tissue maturation compared to the control group and other treatment groups. Moreover, compared to the control, PCL, and PCLU groups, the PE 4:6 group showed narrow scar tissue (a width of 1.39±0.44 mm), total re-epithelialization with a clearly visible stratum corneum, and noticeable wound healing with granular layer and prickle layer in the remodeled tissue around the scar. Contrary to the other groups, the PEU 4:6 group showed fully differentiated dense skin with narrow scars (a width of 1.10±0.24 mm) ((E) of FIG. 8), and dense sebaceous glands, sweat glands and hair follicles, indicating rapid recovery. These results indicate that the UA-integrated PE 4:6 nanofiber scaffold (PEU 4:6) supports the wound healing process and minimizes recovery time while preventing scar formation.

In addition, (D) of FIG. 8 shows photomicrographs of Masson's trichrome-stained tissues of each group. At 21 days after injury, collagen deposition on the scar tissue of the control group was small and thin. However, all nanofiber-treated groups showed relatively dense collagen deposition and thick wavy collagen fibers. Among the various groups, the PEU 4:6 group showed the highest density of collagen deposition of wavy collagen. These findings are similar to the results of H&E staining, indicating that the PEU 4:6-treated group has a rapid upper body recovery ability and that PEU 4:6 accelerates the wound healing process.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A nanofiber scaffold comprising:
polycaprolactone (poly(ε-caprolactone; PCL);
decellularized extracellular matrix (dECM); and
usnic acid (UA).

2. The nanofiber scaffold of claim 1, wherein the nanofiber scaffold is prepared by electrospinning.

3. The nanofiber scaffold of claim 1, wherein the nanofiber scaffold includes the polycaprolactone and the decellularized extracellular matrix in a weight ratio of 8 to 2:2 to 8.

4. The nanofiber scaffold according to claim 1, wherein the nanofiber scaffold is cross-linked.

5. A method of preparing a nanofiber scaffold, the method compising:
mixing polycaprolactone, decellularized extracellular matrix, and usnic acid (phase 1);
preparing a nanofiber mat by electrospinning the mixture (phase 2);
cross-linking the nanofiber mat (phase 3); and
freeze-drying the cross-linked nanofiber mat (phase 4).

6. The method of claim 5, wherein, in phase 1, the polycaprolactone and the decellularized extracellular matrix are mixed in a weight ratio of 8 to 2:2 to 8.

7. The method of claim 5, wherein, in phase 3, the nanofiber mat is impregnated with EDC and NHS in ethanol and cross-linked.

8. A wound dressing comprising the nanofiber scaffold according to claim 1.

9. A wound dressing comprising the nanofiber scaffold according to claim 2.

10. A wound dressing comprising the nanofiber scaffold according to claim 3.

11. A wound dressing comprising the nanofiber scaffold according to claim 4.
